(19) United States
(12) Patent Application Publication
(10) Pub. No.: US 2025/0188506 A1
(43) Pub. Date: Jun. 12, 2025

(54) ANTICOAGULANT HEPARIN-CHONDROITIN CHIMERIC SACCHARIDE MOLECULE AS WELL AS PREPARATION METHOD AND APPLICATION THEREOF

(71) Applicant: SHANDONG UNIVERSITY, Jinan (CN)

(72) Inventors: Chunhui LIU, Jinan (CN); Lin WANG, Jinan (CN); Aohui LIU, Jinan (CN); Qinyu LIU, Jinan (CN); Linhan CUI, Jinan (CN)

(73) Assignee: SHANDONG UNIVERSITY, Jinan (CN)

(21) Appl. No.: 18/967,713

(22) Filed: Dec. 4, 2024

(30) Foreign Application Priority Data
Dec. 6, 2023 (CN) 2023116644760

(51) Int. Cl. C12P 19/44; A61K 31/727; C12N 9/10; C12N 9/90

(52) U.S. Cl. CPC C12P 19/44; A61K 31/727; C12N 9/1051; C12N 9/13; C12N 9/90; C12Y 204/01175; C12Y 204/01219; C12Y 204/01224; C12Y 208/02008; C12Y 208/02023; C12Y 208/0203; C12Y 501/03007

(57) ABSTRACT

An anticoagulant heparin-chondroitin chimeric saccharide molecule as well as a preparation method and application thereof are disclosed. The anticoagulant heparin-chondroitin chimeric saccharide molecule has a structure as shown in formula I. The heparin-chondroitin chimeric saccharide molecule of the present disclosure has potent activities against an Xa factor and IIa, and the activity of the heparin-chondroitin chimeric saccharide molecule can be effectively neutralized by protamine, with a neutralization rate of greater than or equal to 70%. The risk of causing adverse reactions such as fatal HIT is obviously lower than that of enoxaparin and other low-molecular-weight heparins. The heparin-chondroitin chimeric saccharide molecule disclosed by the present disclosure is suitable for the preparation of a safer potent anticoagulant and antithrombotic new drug.

### CROSS-REFERENCE TO RELATED APPLICATIONS

This application claims priority to Chinese Patent Application Ser. No. CN2023116644760 filed on 6 Dec. 2024.

### FIELD OF THE INVENTION

The present disclosure relates to an anticoagulant heparin-chondroitin chimeric saccharide molecule as well as a preparation method and application thereof, belonging to the technical field of biomedicine.

### BACKGROUND OF THE INVENTION

Heparin belongs to a highly sulfated glycosaminoglycan family, and its main products as anticoagulants include unfractionated heparin (UFH; having a weight-average molecular weight of 14000 Da) and chemical/enzymatic partially-depolymerized products, low-molecular-weight heparins (LMWH; having a weight average molecular weight of 3500-6000 Da), extracted from porcine intestinal mucosa. The study showed that UFH and LMWH derived from animal tissues inhibited a coagulation factor Xa by specifically binding a unique pentasaccharide sequence in its saccharide chain (abbreviation: GlcNS/Ac6S-GlcA-GlcNS6S3S-IdoA2S-GlcNS6S) to antithrombin (AT), and had a thrombin (IIa factor) inhibition effect depending on heparin octadecasaccharide containing an AT-binding sequence at a reducing end. Therefore, long-chain UFH has both potent activities against Xa and IIa factors, while LMWH has strong activity against Xa and very low activity against the IIa factor. LMWH has become a preferred anticoagulant drug in clinical practice due to its advantages of a longer half-life in vivo, higher bioavailability, less side effects, safe use, etc., but it has lower artery thrombosis inhibition than that of UFH due to insufficient activity against the IIa factor. In addition, the anticoagulant activity of UFH can be completely neutralized by protamine, while the anticoagulant activity of LMWH can only be partially neutralized.

Although UFH and LMWH are indispensable in clinical practice, the existing animal-derived heparin supply chain is fragile and has a risk of impurity contaminations, and the structural heterogeneity makes it an insurmountable clinical limitation, so a synthetic heparin molecule with defined structure has received a lot of attention and importance. Methyl glycoside of AT-binding pentasaccharide sequence, i.e., fondaparinux sodium (trade name: Arixtra) was completely synthesized by a chemical method and was approved for marketing in 2001 as a potent Xa factor inhibitor. It was the first heparin molecule with a defined structure, but its anticoagulant activity cannot be neutralized by protamine, so its anticoagulant therapy cannot be discontinued at any time. In 2014, Professor Jian Liu published a heparin dodecasaccharide containing a single AT-binding pentasaccharide sequence and four consecutive trisulfate disaccharides (IdoA2S-GlcNS6S), which was also an Xa factor-specific inhibitor. Its anticoagulant activity can be effectively neutralized by protamine, but this molecule had a very short half-life in vivo and also had the possibility to cause fatal heparin-induced thrombocytopenia (HIT).

Based on the research progress and clinical application experience of heparin, the “ideal” anticoagulant heparin should have both potent activities against Xa and IIa factors, be efficiently neutralized for its anticoagulant activity by protamine, with very low or no risk of HIT. It also should have a long half-life in vivo, high bioavailability, few side effects and is safety in use, but the current anticoagulant heparin is more or less defective.

Therefore, it is of great clinical value to design anticoagulant heparin which has both potent activities against Xa and IIa factors, be efficiently neutralized for its anticoagulant activity by protamine and have very low or no risk of HIT, a long half-life in vivo, high bioavailability, few side effects, and safe use.

### SUMMARY OF THE INVENTION

In view of the defects of the prior art, the present disclosure provides an anticoagulant heparin-chondroitin chimeric saccharide molecule as well as a preparation method and application thereof.

Description of terms
AT: antithrombin
IdoA: iduronic acid
GlcA: glucuronic acid
GlcNTFA: N-trifluoroacetylglucosamine
GalNAc: N-acetylgalactosamine
UDP-GlcNTFA: uridine diphosphate-N-trifluoroacetylglucosamine
UDP-Ga1NAc: uridine diphosphate-N-acetylgalactosamine
UDP-G1cA: uridine diphosphate-glucuronic acid
PAPS: 3′-phosphoadenosine-5′-phosphosulfate
KfiA: Escherichia coli K5 N-acetylglucosaminyltransferase
PmHS2: Pasteurella multocida Heparosan synthase 2
KfoC: Escherichia coli K4 chondroitin synthase
NST: N-sulfotransferase
C₅-epi: C₅-isomerase
OST: 2-O-sulfotransferase
6OST: 6-O-sulfotransferase
3OST: 3-O-sulfotransferase

### The Present Disclosure is Implemented by the Following Technical Solutions.

The first object of the present disclosure is to provide an anticoagulant heparin-chondroitin chimeric saccharide molecule.

An anticoagulant heparin-chondroitin chimeric saccharide molecule is formed by connecting the heparin-oligosaccharide chains containing an AT-binding sequence in series with a chondroitin-oligosaccharide chain, and is a compound having a structure shown in formula I or a pharmaceutically acceptable salt thereof;

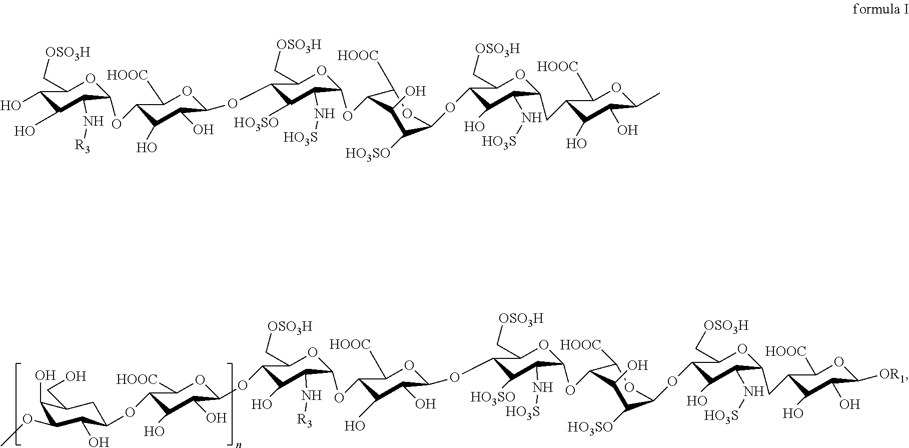

where R₁ is phenyl or substituted phenyl, aromatic heterocycle or substituted aromatic heterocycle, or hydrogen (—H), or alkyl and alkylamine-derived groups with characteristic ultraviolet absorption;
R₂ and R₃ are sulfonic acid groups (—SO₃H) or acetyl groups (—COCH₃); and
n is 1-4.

According to the present disclosure, preferably, substituents of substituted phenyl and substituted aromatic heterocycle are halogen, hydroxyl, nitro or trifluoromethyl.

According to the present disclosure, preferably, the anticoagulant heparin-chondroitin chimeric saccharide molecule is formed by connecting two heparin-oligosaccharide sequences containing AT-binding sequences in series with one non-sulfated chondroitin.

According to the present disclosure, preferably, the anticoagulant heparin-chondroitin chimeric saccharide molecule is selected from any of the following compounds or pharmacologically acceptable salts thereof:

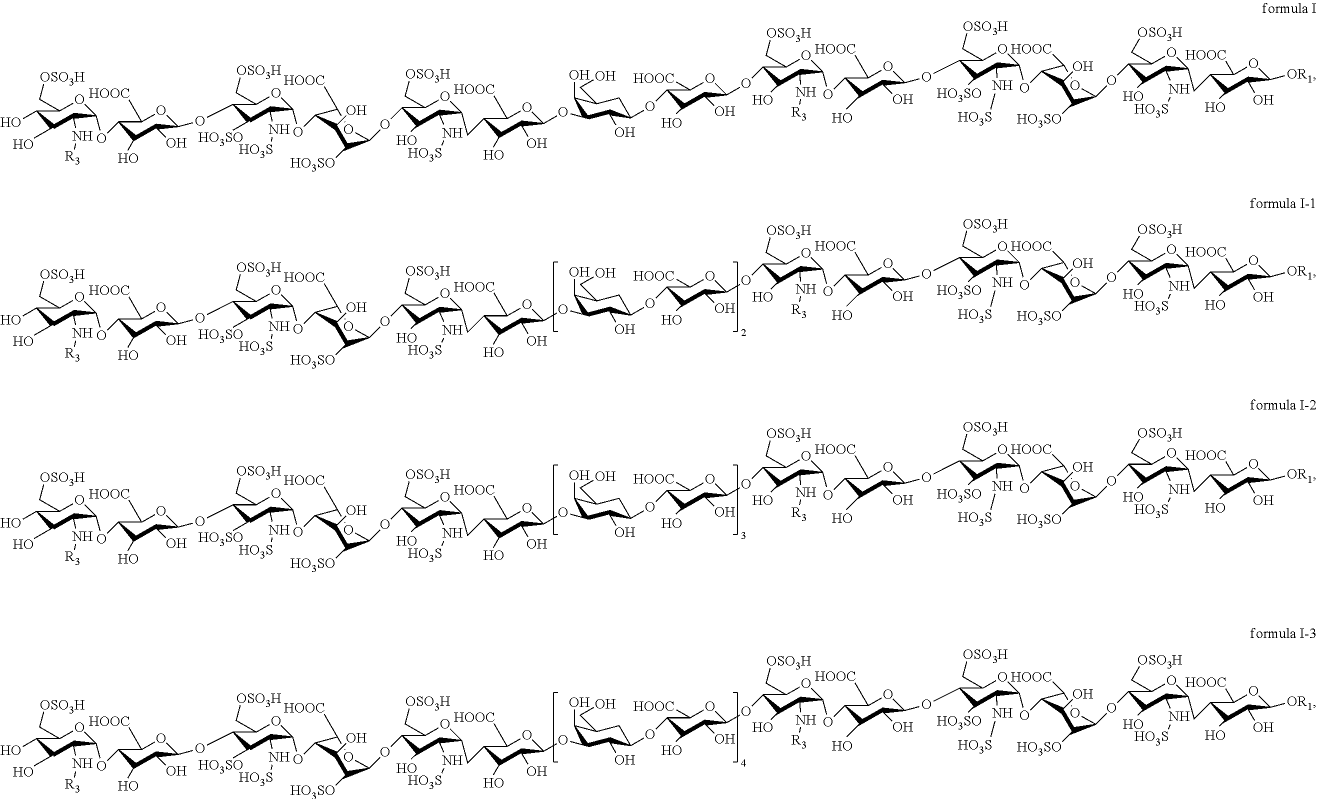

in formulas I-1 to I-4, R₂ and R₃ are sulfonic acid groups (—SO₃H) or acetyl groups (—COCH₃).

The anticoagulant heparin-chondroitin chimeric saccharide molecule provided by the present disclosure has significant activities against an Xa factor and an IIa factor, and the anticoagulant activity can be effectively neutralized by protamine, with a neutralization rate of greater than 70%. The risk of adverse reactions such as HIT is obviously lower than that of animal-derived unfractionated heparin and low-molecular-weight heparin.

The second aspect of the present disclosure is to provide a preparation method of the anticoagulant heparin-chondroitin chimeric saccharide molecule.

The preparation method of the anticoagulant heparin-chondroitin chimeric saccharide molecule is carried out by a chemoenzymatic synthesis strategy, wherein the method includes: by taking a glucuronic acid (GlcA) derivative covalently connected to an R₁ group at its reducing end as a starting substrate, the following steps a and b of saccharide chain extension reactions catalyzed by glycosyltransferases repeated at least once, and in conjunction with two, three or four of steps d, e, f and g which are heparin-saccharide chain chemoenzymatic modification reactions;
at step a, under the catalysis of N-acetylglucosaminyltransferase (KfiA) or Heparosan synthase 2 (PmHS2), with UDP-GlcNTFA or UDP-GlcNAc as a glycosyl donor, a GlcNTFA residue or GlcNAc residue of the glycosyl donor is transferred to a GlcA residue at the non-reducing end of a substrate and linked through an α-1,4 glycosidic bond to obtain an intermediate compound;
at step b, under the catalysis of a PmHS2 enzyme, with UDP-GlcA as a glycosyl donor, a GlcA residue of the glycosyl donor is connected to glucosamine (GlcNTFA or GlcNAc) at the non-reducing end of the substrate through a β-1,4 glycosidic bond to obtain an intermediate compound;
at step c, performing chondroitin synthase (KfoC) catalysis;
at step d, the heparin intermediate is still placed on ice in a mild alkaline aqueous solution, all GlcNTFA residues of a saccharide chain are subjected to trifluoroacetyl (TFA) removal and converted into GlcNH₂, and then into GlcNS under the catalysis of N-sulfotransferase (NST) to obtain an N-sulfated intermediate;
at step e, under the co-catalysis of C₅-isomerase (C₅-epi) and 2-O-sulfotransferase (2OST), a specific GlcA residue in a saccharide chain of the N-sulfated product between two GlcNSs or between GlcNS (non-reducing end) and GlcNAc is converted into 2-O-sulfated iduronic acid (IdoA2S), thereby obtaining an intermediate containing an IdoA2S residue;
at step f, under the co-catalysis of 6-O-sulfotransferase 1 and 6-O-sulfotransferase 3 (6OST1 and 6OST3), 6-OH of all GlcNS or GlcNAc residues of the substrate saccharide chain is subjected to sulfated modification to form GlcNS6S or GlcNAc6S, thereby forming a 6-O-sulfated intermediate; and
at step g, under the catalysis of 3-O-sulfotransferase 1 (3OST1), 3-OH of GlcNS6S in the substrate saccharide chain between GlcA and IdoA2S is sulfated (GlcNS6S3S) to obtain a final target compound.

According to the present disclosure, preferably, step c includes the following steps c1 and c2, wherein
at step c1, under the catalysis of chondroitin synthase KfoC, with UDP-GalNAc as a glycosyl donor, a GalNAc residue of the glycosyl donor is transferred to a GlcA residue at the non-reducing end of the saccharide chain substrate through a 3-1,4 glycosidic bond to obtain an intermediate compound; and
at step c2, under the catalysis of chondroitin synthase KfoC, with UDP-GlcA as a glycosyl donor, a GlcA residue of the glycosyl donor is transferred to a GalNAc residue at the non-reducing end of the saccharide chain substrate through a 3-1,3 glycosidic bond to obtain an intermediate compound.

According to the present disclosure, preferably, a starting substrate is p-nitrophenyl-β-D-glucuronide (GlcA-PNP).

According to the present disclosure, preferably, at step a, N-acetylglucosaminyltransferase (KfiA) and Heparosan synthase 2 (PmHS2) are recombinantly expressed in Escherichia coli; KfiA is derived from Escherichia coli K5; and PmHS2 is derived from Pasteurella multocida.

According to the present disclosure, preferably, at step c, chondroitin synthase (KfoC) is recombinantly expressed in Escherichia coli; and KfoC is derived from Escherichia coli K4.

According to the present disclosure, preferably, at steps a, b and c, a buffer used in an enzyme-catalyzed reaction is 50 mmol/L Tris-HCl; Tris-HCl contains 6 mmol/L MnCl₂ with pH of 7.0-7.5; the reaction temperature is 20° C.-37° C.; the addition amount of the enzyme and the substrate and the reaction time are not limited; the obtained enzymatic reaction solution is purified by reversed-phase C18 or anion-exchange column chromatography to obtain an intermediate compound; and the addition amount of the glycosyl donor is more than 1.2 times the equivalent of the substrate.

According to the present disclosure, preferably, at steps d, e, f and g, heparin-modifying enzymes NST, C₅-epi, 2OST, 6OST1, 6OST3 and 3OST1 are recombinantly expressed by Escherichia coli, yeast or insect cells; heparin-modifying enzymes NST, 2OST, 6OST1, 6OST3 and 3OST1 all use 3′-phosphoadenosine-5′-phosphosulfate (PAPS) as a sulfate-based donor; a buffer catalyzed by each modifying enzyme is 50 mmol/L 2-(N-morpholino)ethanesulfonic acid (MES) with pH of 7.0-7.5; the reaction temperature is 20° C.-37° C.; the addition amount and the reaction time of enzymes and the heparin intermediate substrate are not limited; the obtained reaction solution is purified by anion-exchange column chromatography to obtain a product.

According to the present disclosure, preferably, at steps d, e, f and g, the addition amount of the sulfate-based donor is 1.2-10 times the equivalent of the substrate.

The preparation method of the anticoagulant heparin-chondroitin chimeric saccharide molecule provided by the present disclosure is established on the basis of studying the catalytic activity and substrate specificity of each glycosyltransferase and heparin-modifying enzyme on different chimeric intermediate molecules through repeated experiments.

According to the present disclosure, preferably, the preparation method is selected from one of the following synthesis routes:

No.: I-1, R₂=—SO₃H, the synthesis route is:
(a→b)×2→d→e→a→b→d→c1→c2→f→(a→b)×2→d→e→a→d, or no→f→g (22/21 steps);

No.: I-1, R₂=—COCH₃, the synthesis route is:
(a→b)×3→c1→c2→(a→b)×2→d→e→a→d, or no→f→g (18/17 steps);

No.: I-2, R₂=—SO₃H, the synthesis route is:
(a→b)×2→d→e→a→b→d→(c1→c2)×2→f→(a→b)×2→d→e→a→d, or no→f→g (24/23 steps);

No.: I-2, R₂=—COCH₃, the synthesis route is:
(a→b)×3→(c1→c2)×2→(a→b)×2→d→e→a→d, or no→f→g (20/19 steps);

No.: I-3, R₂=—SO₃H, the synthesis route is:
(a→b)×2→d→e→a→b→d→(c1→c2)×3→f→(a→b)×2→d→e→a→d, or no→f→g (26/25 steps);

No.: I-3, R₂=—COCH₃, the synthesis route is:
(a→b)×3→(c1→c2)×3→(a→b)×2→d→e→a→d, or no→f→g (22/21 steps);

No.: I-4, R₂=—SO₃H, the synthesis route is:
(a→b)×2→d→e→a→b→d→(c1→c2)×4→f→(a→b)×2→d→e→a→d, or no→f→g (28/27 steps); and

No.: I-4, R₂=—COCH₃, the synthesis route is:
(a→b)×3→(c1→c2)×4→(a→b)×2→d→e→a→d, or no→f→g (24/23 steps).

According to the present disclosure, preferably, a preparation method of a chimeric octadecasaccharide molecule composed of two heparin hexasaccharide sequences and one chondroitin hexasaccharide sequence includes the following synthesis route:
(a→b)×2→d→e→a→b→d→(c1→c2)×3→f→(a→b)×2→d→e→a→d→f→g (26 steps).

Specifically, the preparation method of a chimeric octadecasaccharide molecule composed of two heparin hexasaccharide sequences and one chondroitin hexasaccharide sequence includes the following steps:
1) under the catalysis of N-acetylglucosaminyltransferase (KfiA) or Heparosan synthase 2 (PmHS2), with UDP-GlcNTFA as a glycosyl donor, transferring a GlcNTFA residue of the glycosyl donor to GlcA at a non-reducing end of a substrate through an α-1,4 glycosidic bond to obtain a heparin disaccharide backbone intermediate;
2) under the catalysis of a PmHS2 enzyme, with UDP-GlcA as a glycosyl donor, connecting a GlcA residue of the glycosyl donor to GlcNTFA at a non-reducing end of a disaccharide backbone through a β-1,4 glycosidic bond to obtain a heparin trisaccharide backbone intermediate;
3) repeating step 1) and step 2) to extend a saccharide chain to obtain a heparin pentasaccharide backbone intermediate;
4) placing the pentasaccharide backbone intermediate still on ice in a mild alkaline aqueous solution, performing trifluoroacetyl (TFA) removal on all GlcNTFA residues of the saccharide chain, and converting them into GlcNH₂, and then into GlcNS under the catalysis of N-sulfotransferase (NST), thereby obtaining an N-sulfated heparin pentasaccharide intermediate;
5) under the co-catalysis of C₅-isomerase (C₅-epi) and 2-O-sulfotransferase (2OST), converting a specific GlcA residue in a saccharide chain of the N-sulfated heparin pentasaccharide intermediate between two GlcNSs into 2-O-sulfated iduronic acid (IdoA2S) to obtain a heparin pentasaccharide intermediate containing one IdoA2S residue;
6) with the heparin pentasaccharide intermediate containing one IdoA2S residue as a substrate, extending the saccharide chain under the catalysis of KfiA or PmHS2 to obtain a heparin hexasaccharide intermediate, with reaction conditions referring to step 1); with the hexasaccharide intermediate as a substrate, extending the saccharide chain into heptasaccharide by PmHS2 catalysis to obtain a heparin heptasaccharide intermediate, with reaction conditions referring to step 2);
7) placing the heparin heptasaccharide intermediate still on ice in a mild alkaline aqueous solution, performing trifluoroacetyl (TFA) removal on all GlcNTFA residues of the saccharide chain, and converting them into GlcNH₂, and then into GlcNS under the catalysis of N-sulfotransferase (NST), thereby obtaining an N-sulfated heparin heptasaccharide intermediate, with reaction conditions referring to step 4);
8) under the catalysis of chondroitin synthase (KfoC), with UDP-GalNAc as a glycosyl donor, transferring a GalNAc residue of the glycosyl donor to GlcA at the non-reducing end of the substrate through a 3-1,4 glycosidic bond to obtain a heparin-chondroitin chimeric octosaccharide intermediate; under the continuous catalysis of KfoC, with UDP-GlcA as a glycosyl donor, transferring a GlcA residue of the glycosyl donor to a GalNAc residue at a non-reducing end of the chimeric octosaccharide intermediate substrate through a 3-1,3 glycosidic bond, thereby obtaining a heparin-chondroitin chimeric nonasaccharide intermediate;
9) repeating step 8) twice to introduce a chondroitin hexasaccharide sequence to obtain a heparin-chondroitin chimeric tridecasaccharide intermediate;
10) under the co-catalysis of 6-O-sulfotransferases 1 and 3 (6OST1 and 6OST3), performing sulfated modification on 6-OH of all GlcNS residues of the N-sulfated heparin heptasaccharide intermediate containing one IdoA2S residue to form GlcNS6S, thereby obtaining a 6-O-sulfated heparin-chondroitin chimeric tridecasaccharide intermediate;
11) referring to step 1) and step 2), extending a saccharide chain of the heparin-chondroitin chimeric tridecasaccharide intermediate to obtain a heparin-chondroitin chimeric pentadecasaccharide intermediate; continuously referring to step 1) and step 2), extending the saccharide chain to obtain a heparin-chondroitin chimeric heptadecasaccharide intermediate containing a heparin backbone saccharide chain at a non-reducing end;
12) referring to step 4, performing trifluoroacetyl (TFA) removal on all GlcNTFA residues of the heparin chain at a non-reducing end of the heparin-chondroitin chimeric heptadecasaccharide intermediate, and converting them into GlcNH₂, and then into GlcNS under the catalysis of NST, thereby obtaining a heparin-chondroitin heptadecasaccharide intermediate containing an N-sulfated heparin chain at a non-reducing end;
13) under the co-catalysis of C₅-isomerase (C₅-epi) and 2-O-sulfotransferase (2OST), converting a specific GlcA residue in a heparin saccharide chain at a non-reducing end in chimeric pentasaccharide between two GlcNSs into IdoA2S, thereby obtaining a heparin-chondroitin chimeric pentasaccharide intermediate containing two IdoA2S residues;
14) with UDP-GlcNTFA as a glycosyl donor, extending a saccharide chain under the catalysis of KfiA or PmHS2 to obtain a heparin-chondroitin chimeric octadecasaccharide intermediate, with reaction conditions referring to step 1); referring to step 4, modifying a newly introduced GlcNTFA residue of the chimeric octadecasaccharide intermediate into GlcNS;
15) under the co-catalysis of 6-O-sulfotransferases 1 and 3 (6OST1 and 6OST3), performing sulfated modification on 6-OH of a GlcNS residue of a heparin saccharide chain at a non-reducing end of the chimeric octadecasaccharide intermediate into GlcNS6S, thereby obtaining a heparin-chondroitin chimeric octadecasaccharide intermediate whose heparin chain is 6-O-sulfated completely; and
16) under the catalysis of 3-O-sulfotransferase 1 (3OST1), sulfating 3-OH of a GlcNS6S residue of a heparin saccharide chain in the chimeric octadecasaccharide intermediate between GlcA and IdoA2S to obtain a target compound I-3 formed by connecting two heparin hexasaccharide sequences containing AT-binding sequences with one non-sulfated chondroitin hexasaccharide sequence, wherein R₂ and R₃ are sulfonic acid groups (—SO₃H).

The third object of the present disclosure is to provide the application of the anticoagulant heparin-chondroitin chimeric saccharide molecule in the preparation of an anticoagulant and anti-thrombotic drug.

An anticoagulant and anti-thrombotic drug includes the anticoagulant heparin-chondroitin chimeric saccharide molecule and one or more pharmaceutically acceptable carriers or excipients, wherein a ratio of the chimeric glycosyl molecule to the carriers or excipients is not limited.

Those that are not specifically described in the present disclosure, such as dosages and reaction conditions, are described according to the existing technologies in the art.

### The Present Disclosure has the Following Technical Features and Advantages.

1. The anticoagulant heparin-chondroitin chimeric saccharide molecule of the present disclosure is formed by connecting a heparin-oligosaccharide chain containing an AT-binding sequence in series with a chondroitin oligosaccharide chain, which is the first glycosaminoglycan molecule with a novel structure; based upon the determination via chromogenic substrate assay, the anticoagulant heparin-chondroitin chimeric saccharide molecule of the present disclosure has significant activity against the Xa factor; and it is confirmed for the first time that the chondroitin saccharide chain in the chimera does not interfere with independent activation of AT by two heparin oligosaccharide chains containing AT-binding sequences to inactivate the Xa factor, and the median inhibition molar concentration (ICso) for the inhibition of the Xa factor is significantly lower than that of positive control fondaparinux containing a single AT-binding sequence and a heparin dodecasaccharide molecule reported by Jian Liu (Nat Chem Biol, 2014, 10:248-50).

2. Based on the verification via chromogenic substrate assay, the anticoagulant heparin-chondroitin chimeric saccharide molecule of the present disclosure has significant activity against the IIa factor, that is, is a potent anticoagulant new molecule with activities against both the Xa factor and the IIa factor.

3. The specific anticoagulant activity of the novel anticoagulant heparin-chondroitin chimeric saccharide molecule of the present disclosure can be effectively neutralized by protamine, with a neutralization rate of >70%, which is comparable to that of the reported heparin dodecasaccharide molecule (Nat Chem Biol, 2014, 10:248-50; Sci Transl Med, 2017, 9: eaan5954) and superior to that of animal-derived LMWH enoxaparin, while the activity against the Xa factor of commercially available fondaparinux is almost completely not neutralized by protamine. Therefore, the heparin-chondroitin chimera of the present disclosure still has similar characteristics to a long-chain heparin molecule, and can produce a high-affinity interaction with protamine to be neutralized despite a non-sulfated chondroitin oligosaccharide sequence is in the middle of the saccharide chain.

4. The capability of the heparin-chondroitin chimeric saccharide molecule of the present disclosure to form a macromolecular complex with a platelet factor 4 (PF4) is significantly lower than that of enoxaparin, which confirms that the risk of HIT caused by the heparin-chondroitin chimera is significantly lower than that of enoxaparin; and meanwhile, the heparin-chondroitin chimera of the present disclosure has pharmacokinetic characteristics such as a long half-life in vivo and high bioavailability.

5. The heparin-chondroitin chimeric saccharide molecule of the present disclosure can be used to prepare a novel safer anticoagulant and anti-thrombotic drug with strong activities against Xa and IIa factors, which has an important clinical value.

### BRIEF DESCRIPTION OF THE DRAWINGS

FIG. 1A is a high-performance liquid chromatogram of a heparin-chondroitin chimeric saccharide molecule I-1 prepared in Example 2;

FIG. 1B is a mass spectrum of a heparin-chondroitin chimeric saccharide molecule I-1 prepared in Example 2;

FIG. 1C is a high-performance liquid chromatogram of I-3 prepared in Example 3;

FIG. 1D is a mass spectrum of I-3 prepared in Example 3;

FIG. 2A is ¹H NMR spectra of the heparin-chondroitin chimeric saccharide molecules I-1 prepared in Examples 2 and 3;

FIG. 2B is ¹H NMR spectra of the heparin-chondroitin chimeric saccharide molecules I-3 prepared in Examples 2 and 3;

FIG. 3A is a standard curve of anti-FXa potency of the heparin-chondroitin chimeric saccharide molecules I-1 and I-3 prepared in Examples 2 and 3;

FIG. 3B is a standard curve of anti-FIIa titers of the heparin-chondroitin chimeric saccharide molecules I-1 and I-3 prepared in Examples 2 and 3;

FIG. 4 shows neutralizing effects of protamine on the anticoagulant activities in vitro of the heparin-chondroitin chimeric saccharide molecules I-1 and I-3 prepared in Examples 2 and 3; and

FIG. 5A is an SPR diagram determined for the heparin-chondroitin chimeric saccharide molecule I-1 prepared in Examples 2 and 3;

FIG. 5B is an SPR diagram determined for the heparin-chondroitin chimeric saccharide molecule I-3 prepared in Examples 2 and 3;

FIG. 5C is a particle size diagram determined for the heparin-chondroitin chimeric saccharide molecule I-1 prepared in Examples 2 and 3;

FIG. 5D is a particle size diagram determined for the heparin-chondroitin chimeric saccharide molecule I-3 prepared in Examples 2 and 3.

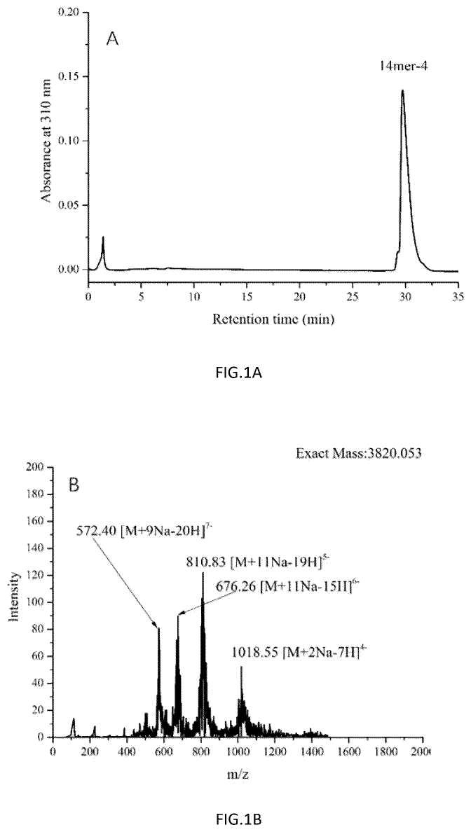

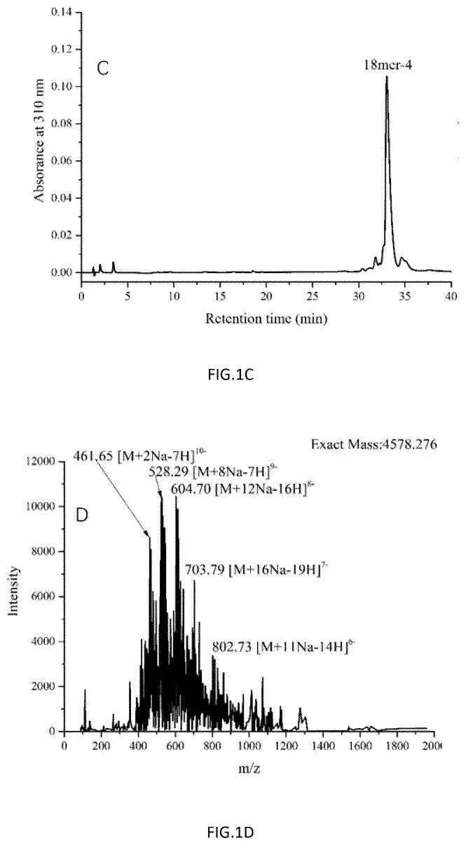

### DETAILED DESCRIPTION OF THE EMBODIMENTS

The present disclosure will be further described and understood below in conjunction with specific examples, which are not intended to limit the protection scope of the present disclosure. The drugs and reagents involved in the examples, unless otherwise specified, are ordinary commercially available products.

Commercialized kits are commercially available products in the prior art.

### EXAMPLE 1: Chemoenzymatic Synthesis of Heparin Heptasaccharide Intermediate Containing Single IdoA2S Residue

500 mg of GlcA-PNP was dissolved in 100 mL of water, added with 10 mL of Tris-HCl solution (1 mol/L, pH=7.5), 1.2 times the equivalent of UDP-GlcNTFA and 0.6 mL of MnCl₂ (2 mol/L) solution, and supplemented with purified water to 194 mL. The pH was adjusted to about 7.5. 6 mg of KfoC enzyme was added. The reaction was carried out in a water bath at 37° C. for 12 h. The reaction progress was detected by using PAMN-HPLC, with detection wavelengths of 260 nm and 310 nm. If the reaction process was slow, the reaction system could be expanded by 10 mL, supplemented with 2 mg of enzyme, and continued to react for 10 h until the reaction yield was greater than 95%. The pH was adjusted to 2-3 by trifluoroacetic acid and the reaction was terminated. The reaction product was stored at −20° C. After being thawed at 30° C., the reaction solution was filtered with a 0.22 m filter membrane to remove a protein, and then purified with a C18 chromatography column (3.0×50 cm) under the following purification conditions: isocratic elution was performed for 30 min with a solution A (20% methanol aqueous solution, containing 0.1% trifluoroacetic acid), and then isocratic elution was performed with a solution B (80% methanol aqueous solution, containing 0.1% trifluoroacetic acid), with a flow rate of 5 mL/min and detection wavelengths of 260 nm and 310 nm. A target peak component was collected under 310 nm detection, which was a disaccharide backbone intermediate 2mer-1. The obtained 2mer-1 was placed in 200 mL of 50 mmol/L Tris-HCl buffer (containing 6 mmol/L MnCl₂, pH=7.2), added with 1.2 times the equivalent of UDP-GlcA and 5 mL of PmHS2 enzyme at the same time and stirred overnight at room temperature. The reaction was detected by PAMN-HPLC until the yield was greater than or equal to 95%. The reaction product was purified with a C18 chromatography column to obtain a trisaccharide backbone intermediate 3mer-1. With 3mer-1 as a substrate, the above KfiA and PmHS2 reactions were repeated to obtain a pentasaccharide backbone intermediate 5mer-1, the purity of 5mer-1 was 85% measured by PAMN-HPLC, and the molecular weight of 5mer-1 was 1181.09 Da measured by ESI-MS, which was consistent with a theoretical value.

400 mg of 5mer-1 was dissolved in 100 mL of water, added with a 0.5 mol/L LiOH solution drop by drop to pH=12, continued to react for 1 h under stirring, and subjected to TFA removal, and the reaction process was detected by PAMN-HPLC. After the reaction was terminated, the pH was adjusted to neutral with glacial acetic acid. A 1 mol/L MES solution (pH=7.5) was added to make the final concentration be 50 mmol/L, added with 3 times the equivalent of PAPS and 4 mg of NST enzyme at the same time, and stirred overnight at room temperature. The reaction was detected by PAMN-HPLC. When the reaction yield was greater than 98%, the pH was adjusted to 4-5 with acetic acid, and the reaction was terminated. Centrifugation was performed at 8000 r/min for 15 min and a precipitate was removed. A supernatant was filtered with a 0.22 m filter membrane to remove a protein, purified with a Q Sepharose column (15×1.5 cm), eluted according to the following conditions: eluted with a 0-60% solution B (containing 50 mmol/L NaOAc, and 1 mol/L NaCl, pH=5) within 200 min, with a flow rate of 3 mL/min, and equilibrated with a solution A (50 mmol/L NaOAc, pH=5) before loading, with detection wavelengths of 260 nm and 310 nm. A target peak component was collected under 310 nm detection, and the purity of the product was detected by PAMN-HPLC. After a sample was concentrated by rotary evaporation, the obtained sample solution was dialysed and desalted with a dialysis bag having molecular weight cut-off (MWCO) of 100 Da to obtain an N-sulfated heparin pentasaccharide (5mer-3) solution, the purity of this solution was 95% measured by PAMN-HPLC, and the molecular weight of this solution was 1149.17 Da measured by ESI-MS, which was consistent with a theoretical value.

350 mg of N-sulfated pentasaccharide (5mer-3) was dissolved in 100 mL of water, and added with 9 mL of MES solution (1 mol/L, pH=7.5) and 0.18 mL of 2 mol/L CaCl₂. The pH was adjusted to about 7.5. Purified water was supplemented to 180 mL, and then added with 10 mg of a C₅-epi enzyme. After the reaction was carried out in a water bath at 37° C. for 1 h, the reaction system was equidistantly expanded by 20 mL, added with 1.5 times the equivalent of PAPS and then with 12 mg of 2OST enzyme, and reacted in a water bath at 30° C. overnight. The reaction process was monitored by PAMN-HPLC, and an enzyme or PAPS were added as needed till the reaction was ended, and the reaction solution was purified with a Q-Sepharose strong anion column (30×1.6 cm) to obtain a product 5mer-4. The purity of 5mer-4 was 95% measured by PAMN-HPLC, and the molecular weight of 5mer-4 was 1129.46 Da measured by ESI-MS, which was consistent with a theoretical value.

The above KfiA, PmHS2 and NST reactions were repeated to obtain a heparin heptasaccharide intermediate 7mer-2 containing a single IdoA2S residue, the purity of 7mer-2 was 98%, and the molecular weight of 7mer-2 was 1645.54 Da verified by ESI-MS, which was consistent with a theoretical molecular weight.

A synthesis route of 7mer-2 was shown in formula II as follows:

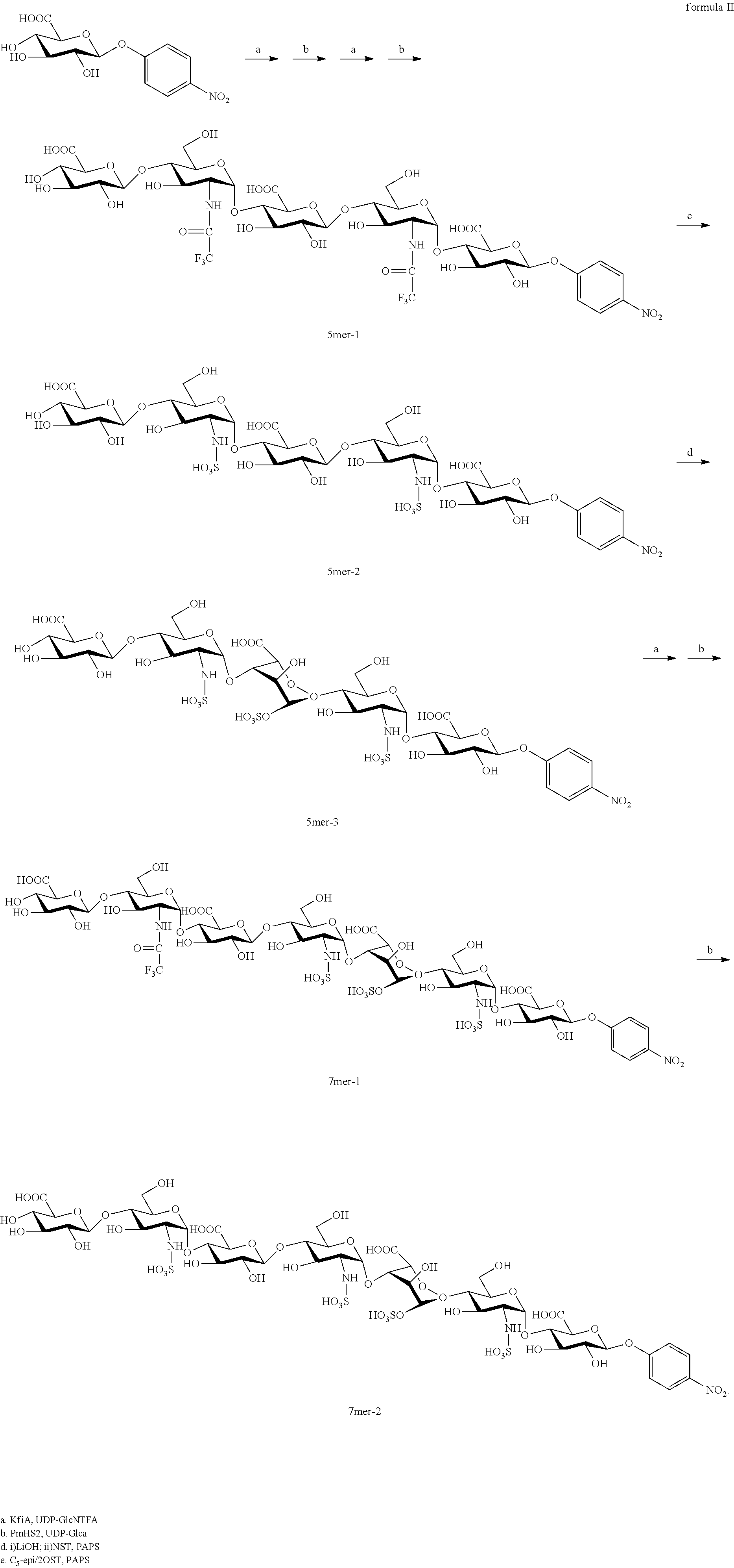

### EXAMPLE 2: Chemoenzymatic Synthesis of Heparin-Chondroitin Chimeric Tetradecasaccharide Containing Double at-Binding Sequences

330 mg of heparin heptasaccharide intermediate 7mer-2 containing one IdoA2S residue prepared in Example 1 was dissolved with 100 mL of water, and added with 10 mL of MES solution (1 mil/L, pH=7.5), and then with 3 times the equivalent of PAPS. The pH was adjusted to about 7.5. Purified water was supplemented to 180 mL, finally added with 10 mg of each of 6OST1 and 6OST3 enzymes, and reacted in a water bath at 30° C. for 12 h. The reaction process was monitored by PAMN-HPLC, with detection wavelengths of 260 nm and 310 nm, until the reaction yield was greater than 99%. The pH of the reaction solution was adjusted to 4-5 with acetic acid till the reaction was terminated, and the reaction solution was stored at −20° C. After being thawed, the reaction solution was purified with a Q Sepharose chromatography column (30×1.5 cm). The purity of the purified 7mer-3 was 92%, and the molecular weight of 7mer-3 was 1885.92 Da verified by ESI-MS, which was consistent with a theoretical molecular weight.

Then, 230 mg of 7mer-3 was dissolved in 100 mL of water, added with 10 mL of Tris-HCl solution (1 mol/L, pH=7.5), 1.5 times the equivalent of UDP-GalNAc and 0.6 mL of MnCl₂ (2 mol/L) solution, and supplemented with purified water to 194 mL. The pH was adjusted to 7.5. 6 mg of KfiA enzyme was added. The reaction was carried out in a water bath at 37° C. for 12 h. The reaction process was monitored by PAMN-HPLC, and the reaction solution was purified with a Q-Sepharose chromatography column (30×1.5 cm) to obtain 8mer-1. A glycosyl donor was replaced with UDP-GlcA. A saccharide chain was further extended under the continuous catalysis of an KfoC enzyme to obtain 9mer-1, the purity of the purified 9mer-1 was 92%, and the molecular weight of 9mer-1 was 2264.64 Da verified by ESI-MS, which was consistent with a theoretical molecular weight.

According to Example 1, with UDP-GlcNTFA as a glycosyl donor, a saccharide chain was extended under the catalysis of KfiA. Next, with UDP-GlcA as a glycosyl donor, the saccharide chain was further extended under the catalysis of a PmHS2 enzyme. Then, the saccharide chain was enzymatically extended alternately by KfiA (glycosyl donor UDP-GlcNTFA) and PmHS2 (glycosyl donor UDP-GlcA) until 13mer-1 was formed, which was then purified by a Q Sepharose chromatography column (1×20 cm). Then, the purified product was subjected to N-sulfated modification by performing LiOH treatment for trifluoroacetyl removal and NST catalysis to obtain heparin undecasaccharide 13mer-2. The purity of the purified 13mer-2 was 87%, and the molecular weight of the purified 13mer-2 was 3099.20 Da verified by ESI-MS, which was consistent with a theoretical molecular weight.

After 13mer-2 was obtained, under the co-catalysis of C₅-isomerase (C₅-epi) and 2-O-sulfotransferase (2OST), specific GlcA in N-sulfated tridecasaccharide between two GlcNSs was converted to 2-O-sulfated iduronic acid (IdoA2S). After the reaction was performed for certain time, the reaction solution was purified by a Q Sepharose chromatography column (1×20 cm) to obtain 13mer-3 (tridecasaccharide containing two IdoA2S residues). The purity of the purified 13mer-3 was 60%, and the molecular weight of the purified 13mer-3 was 3179.22 Da verified by ESI-MS, which was consistent with a theoretical molecular weight.

14mer-1 was obtained by connecting GlcNTFA under the catalysis of a KfiA enzyme. Trifluoroacetyl on GlcNTFA was chemically removed. Then, 14mer-2 was obtained by performing N-sulfated modification under the action of an NST enzyme. Under the co-catalysis of a 6OST1 enzyme and a 6OST3 enzyme, 14mer-3 was obtained by performing 6-O-sulfated modification. After 14mer-3 was obtained, in the case of only enzyme removal but no purification, 2.5 mL of MES solution (1 mol/L, pH=7.5) was supplemented, and then added with 3 times the equivalent of PAPS. The pH was adjusted to about 7.5. Purified water was supplemented to 146 mL, finally added with 4 mg of 3OST1 enzyme, and reacted in a water bath at 30° C. for 12 h. The reaction process was monitored by Q-column-HPLC until the reaction yield was greater than 95%. Chimeric molecule tetradecasaccharide I-1 formed by connecting a chondroitin disaccharide backbone in series with two AT-binding sequences is obtained by performing 3-O-sulfated modification. After purification, the purity of the purified I-1 was 98%, and the molecular weight of the purified I-1 was 3660.08 Da verified by ESI-MS, which was consistent with a theoretical molecular weight.

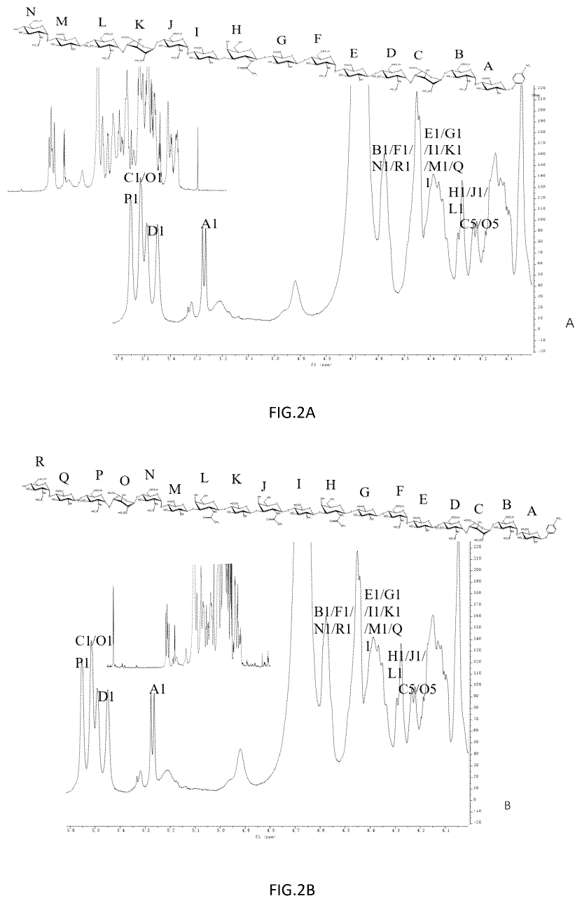

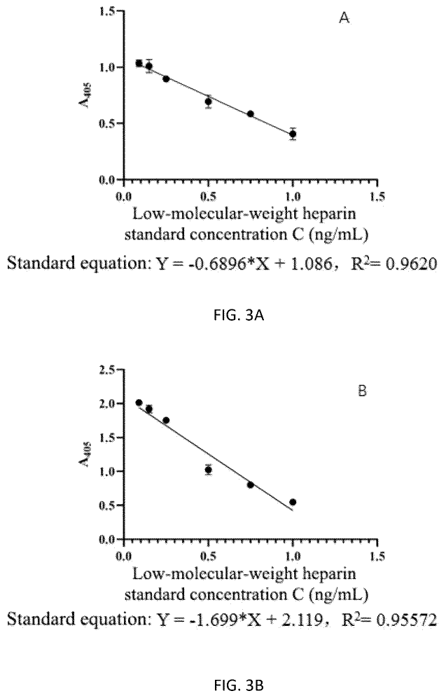

A ¹H NMR (600 MHz, D₂O) spectrum of I-1 was shown in FIG. 2A. As can be seen from FIG. 2A, the structure was as expected.

A synthesis route of the chimeric molecule tetradecasaccharide I-1 was shown in formula III as follows:

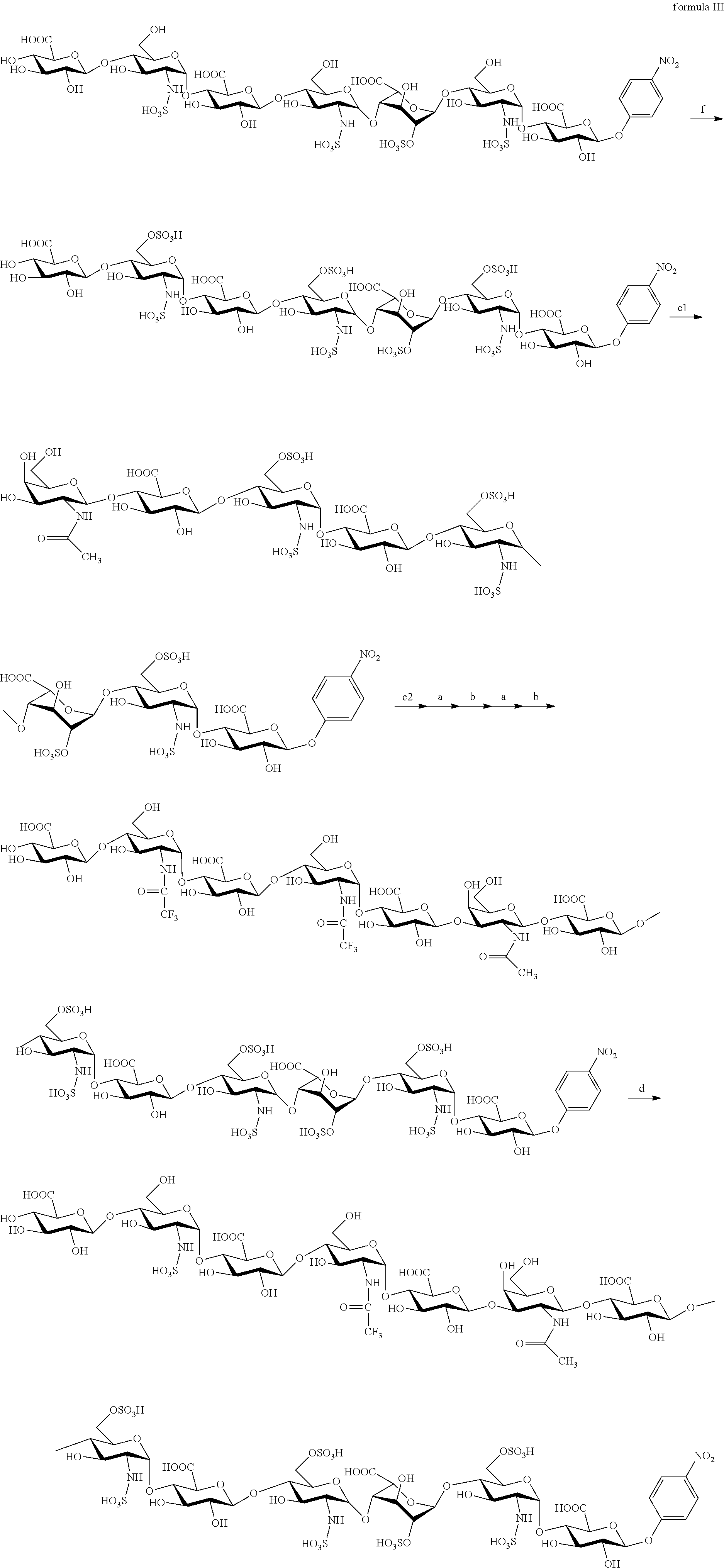

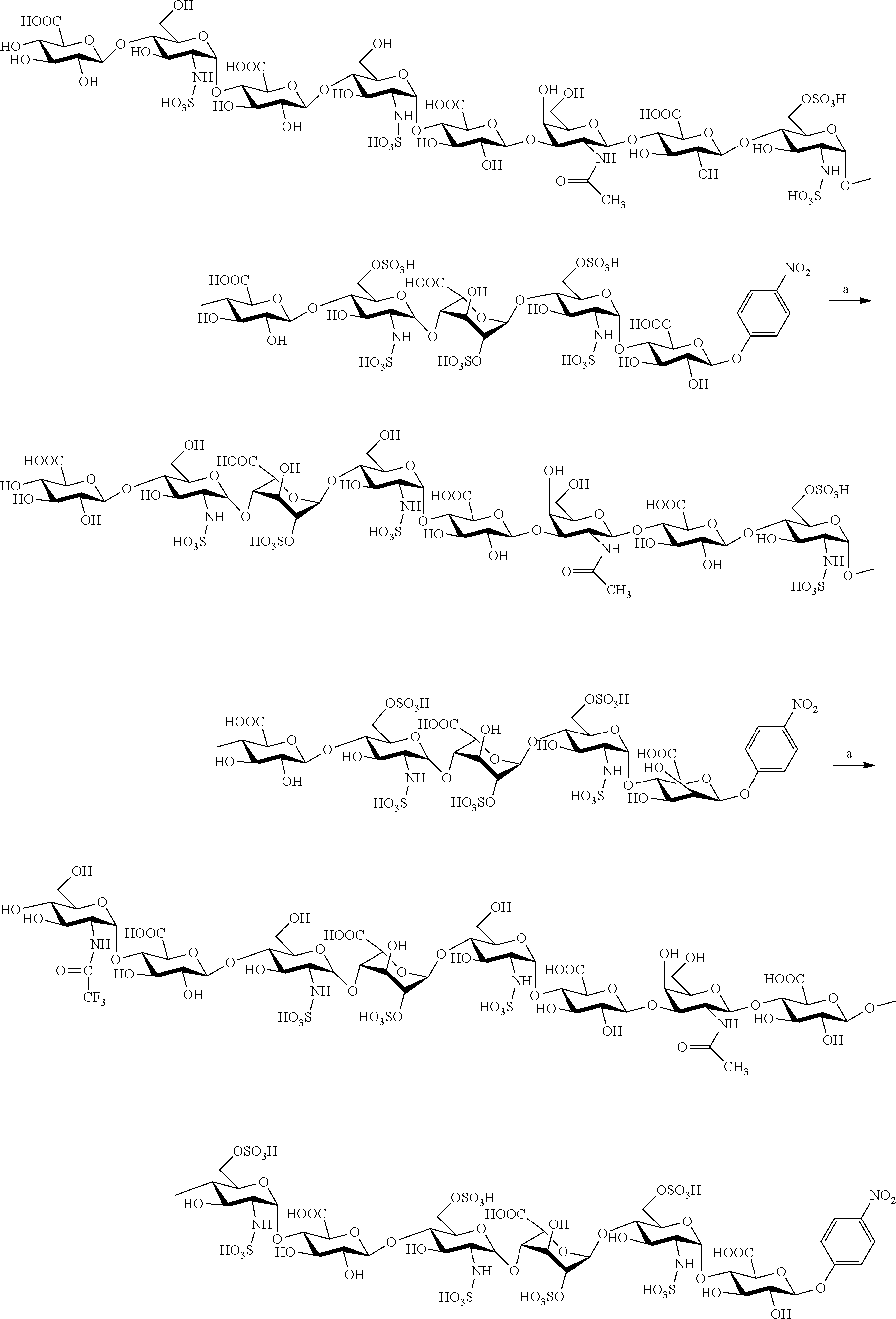

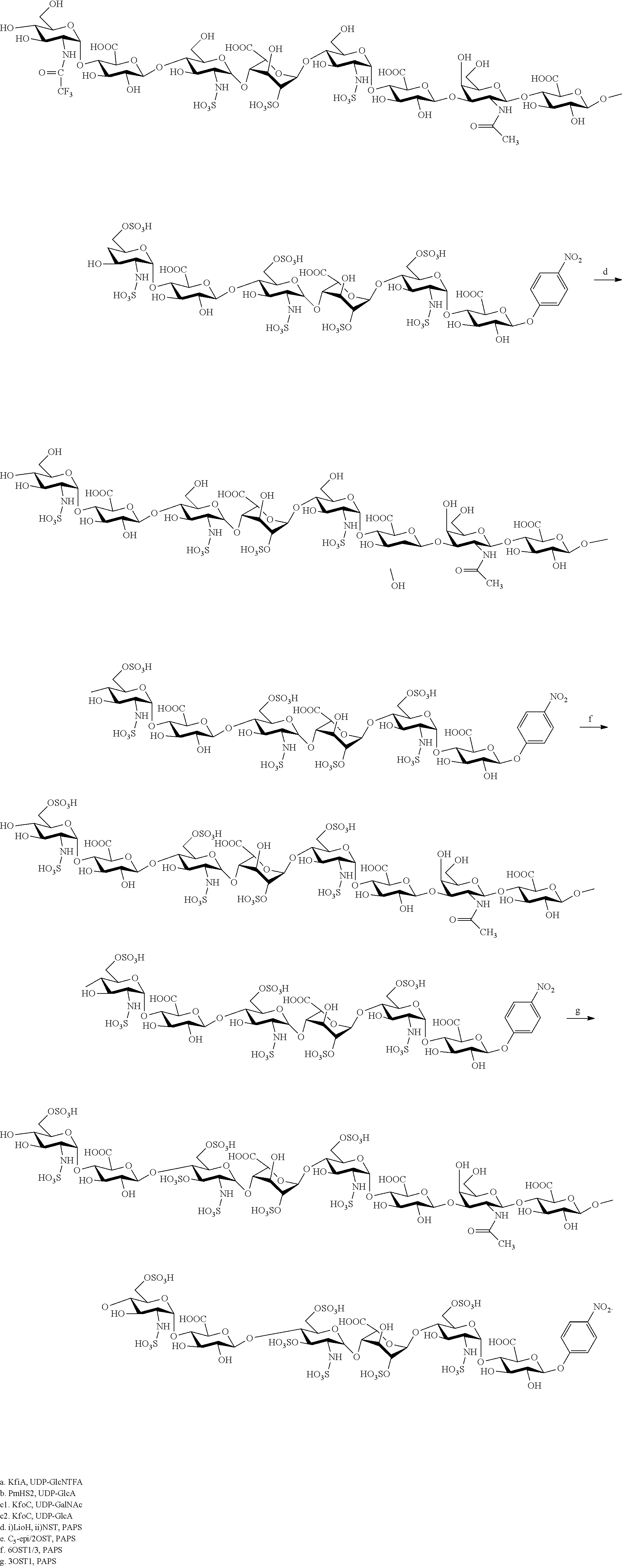

### EXAMPLE 3: Chemoenzymatic Synthesis of Heparin-Chondroitin Chimeric Octadecasaccharide Containing Double AT-Binding Sequences

According to experience and preliminary experiments of Example 2, the synthesis route was optimized and adjusted, which not only avoided the isomerization of eighth monosaccharide GlcA in the saccharide chain, but also solved the problem of low reaction efficiency of the extended saccharide chain caused by 6-O-sulfated modification.

That is, GalNAc and GlcA were alternately added to a non-reducing end of a heparin heptasaccharide intermediate 7mer-2 containing one IdoA2S residue prepared in Example 1 first under the catalysis of a KfoC enzyme so as to introduce six chondroitin backbones. The obtained chondroitin-heparin tandem molecule 13mer-4 was subjected to 6-O-sulfated modification on a heparin sequence in the saccharide chain to obtain 13mer-5 under the catalysis of 6OST1 and 6OST3 enzymes, the purity of the purified 13mer-5 was 92%, and the molecular weight of 13mer-5 was 3023.50 Da verified by ESI-MS, which was consistent with a theoretical molecular weight. Then, GlcNTFA and GlcA were connected under the alternate catalysis of a KfiA enzyme and a PmHS2 enzyme to obtain 17mer-1, the purity of the purified 17mer-1 was 94%, and the molecular weight of the purified 17mer-1 was 3889.40 Da verified by ESI-MS, which was consistent with a theoretical molecular weight. After trifluoroacetyl on GlcNTFA was chemically removed, and then subjected to N-sulfated modification under the action of an NST enzyme to obtain 17mer-2, the purity of the purified 17mer-2 was 97%, and the molecular weight of 17mer-2 was 3856.96 Da verified by ESI-MS, which was consistent with a theoretical molecular weight. Under the co-catalysis of C₅-epi and 2OST enzymes (insect cell expression system), specific GlcA was subjected to Cₛ isomerization and 2-O-sulfated modification to obtain 17mer-3, the purity of the 17mer-3 purified by a C18 column was 82%. 18mer-1 was obtained by adding GlcNTFA under the catalysis of a KfiA enzyme. Trifluoroacetyl on GlcNTFA was chemically removed, and then subjected to N-sulfated modification under the action of an NST enzyme to obtain 18mer-2. After purification, the purity of the purified 18mer-2 was 90%, and the molecular weight of 18mer-2 was 4178.03 Da verified by ESI-MS, which was consistent with a theoretical molecular weight. Under the joint action of a 6OST1 enzyme and a 6OST3 enzyme, 18mer-3 was obtained by performing 6-O-sulfated modification. Finally, under the action of a 3OST-1 enzyme, a target compound I-3 was obtained by 3-O-sulfated modification, the purity of the purified I-3 was 90%, and the molecular weight of I-3 was 4577.50 Da verified by ESI-MS, which was consistent with a theoretical molecular weight.

A ¹H NMR (600 MHz, D₂O) spectrum of I-3 was shown in FIG. 2B. As can be seen from FIG. 2B, the structure was as expected.

Its synthesis route was shown in formula IV as follows:

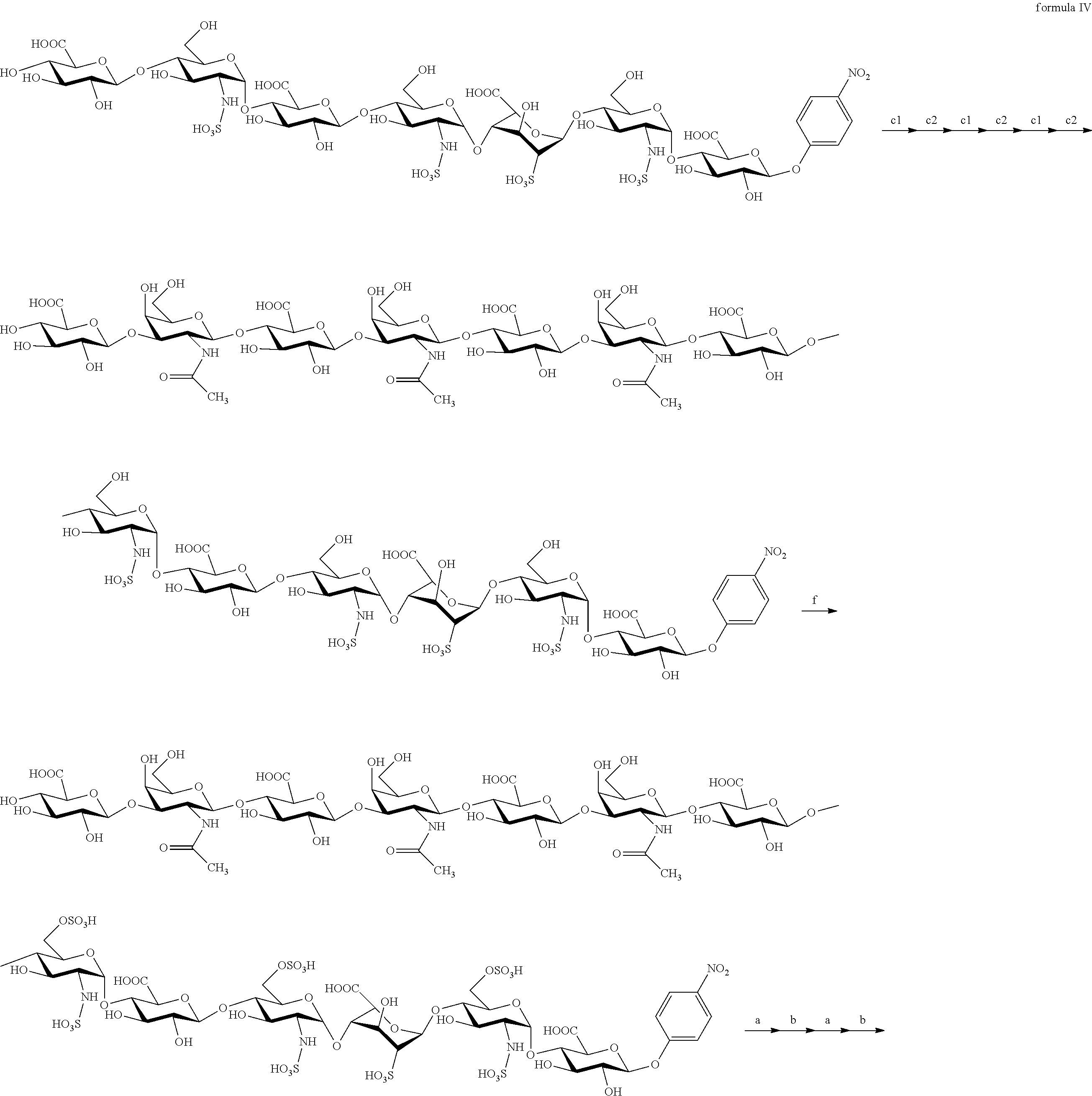

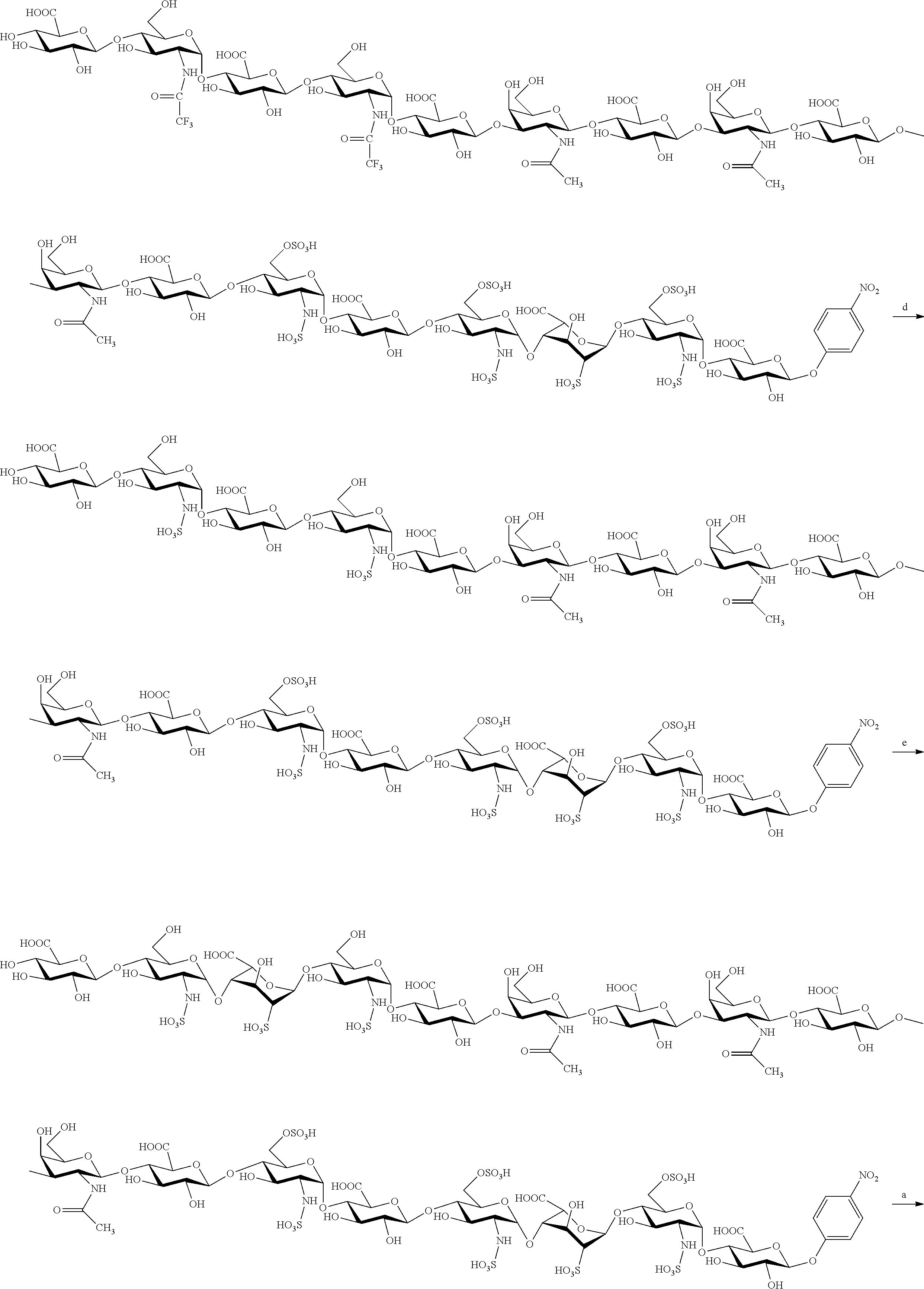

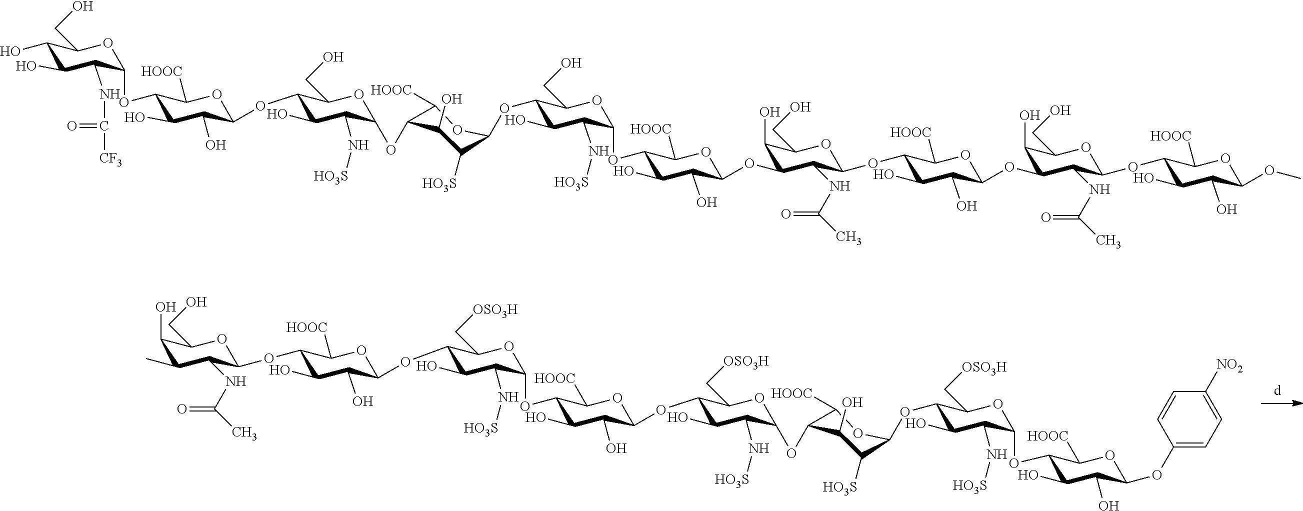

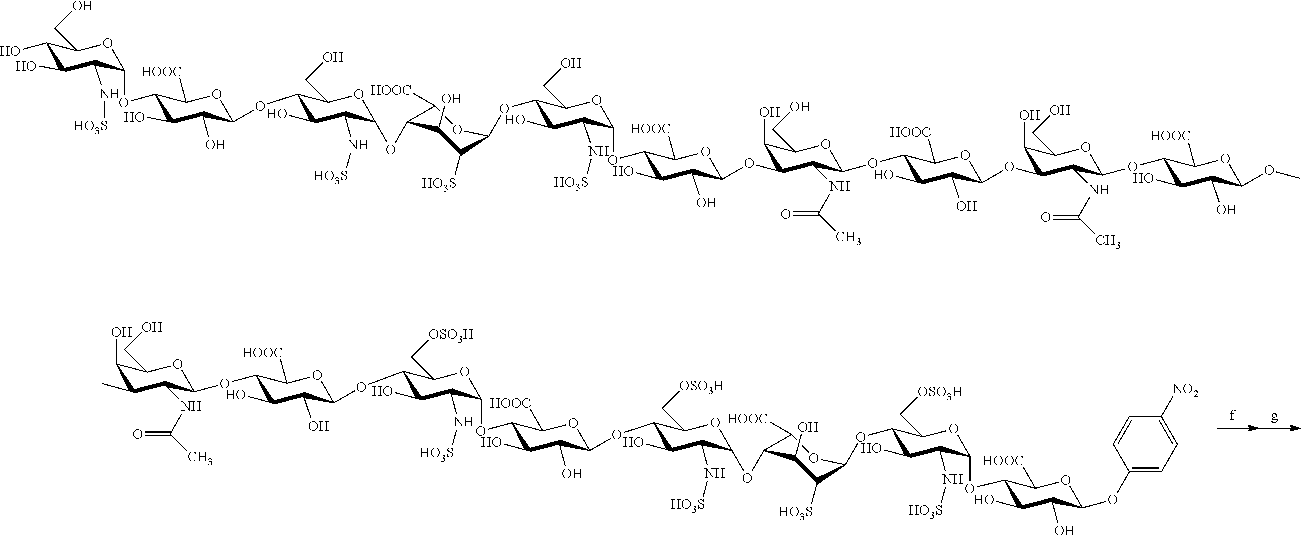

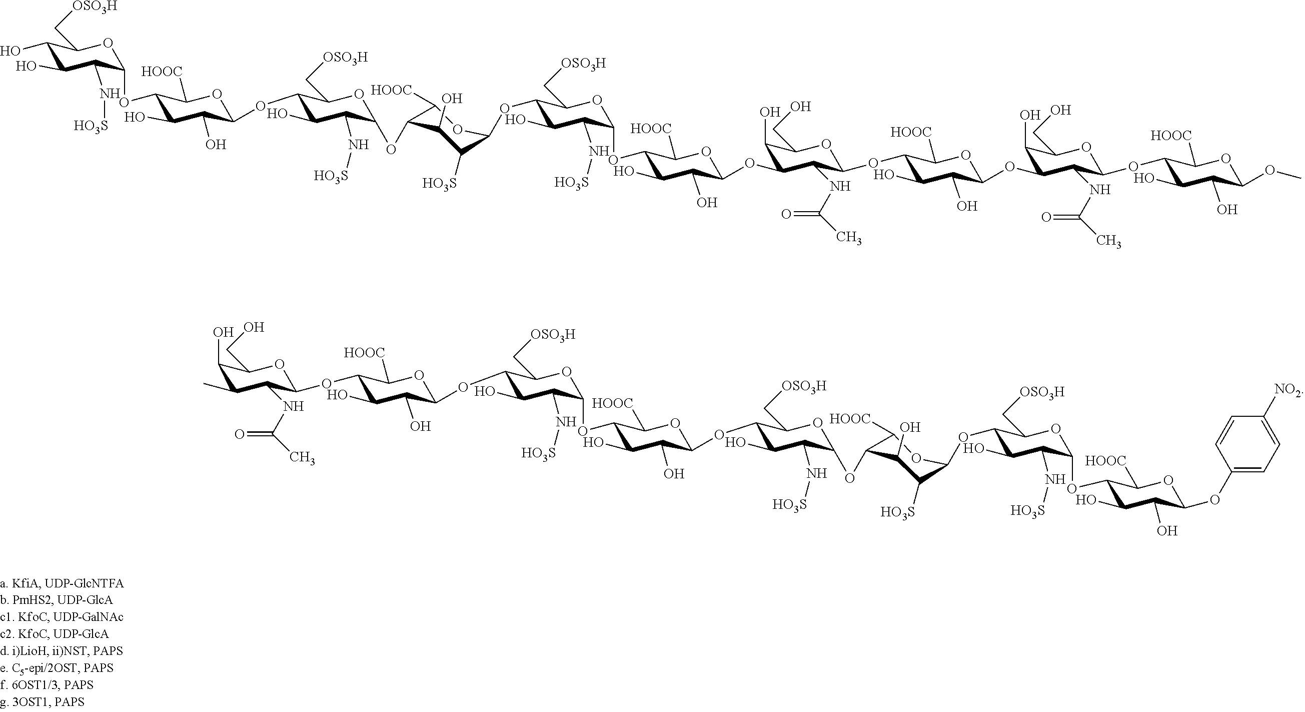

### EXAMPLE 4: In-Vitro Determination of Anticoagulant Activities of Heparin-Chondroitin Chimeras I-1 and I-3

Anti-FXa activity titers of the heparin-chondroitin chimeras I-1 and I-3 prepared in Example 2 and example 3 of the present disclosure were measured by using a commercial kit and chromogenic substrate assay were 798.02 IU/mg and 913.67 IU/mg, and the anti-Xa activity titer of enoxaparin was 101.26 IU/mg measured under the same conditions. It can be seen that the anti-Xa activity titers of the heparin-chondroitin chimeras I-1 and I-3 prepared in the present disclosure were much higher than that of commercially available enoxaparin. Anti-IIa activity titers of the heparin-chondroitin chimeras I-1 and I-3 prepared in the present disclosure were measured by using chromogenic substrate assay were 1.88 IU/mg and 218.13 IU/mg, and the anti-Ia activity titer of enoxaparin was 19.56 IU/mg measured under the same conditions. Therefore, the chimeric molecule I-1 prepared in the present disclosure was a heparin compound with high anti-Xa activity, and I-3 was an anticoagulant heparin compound with both high-efficiency anti-Xa activity and anti-Ia activity.

### EXAMPLE 5: Determination of Neutralizing Effects of Protamine on Anticoagulant Activities of Heparin-Chondroitin Chimeras I-1 and I-3

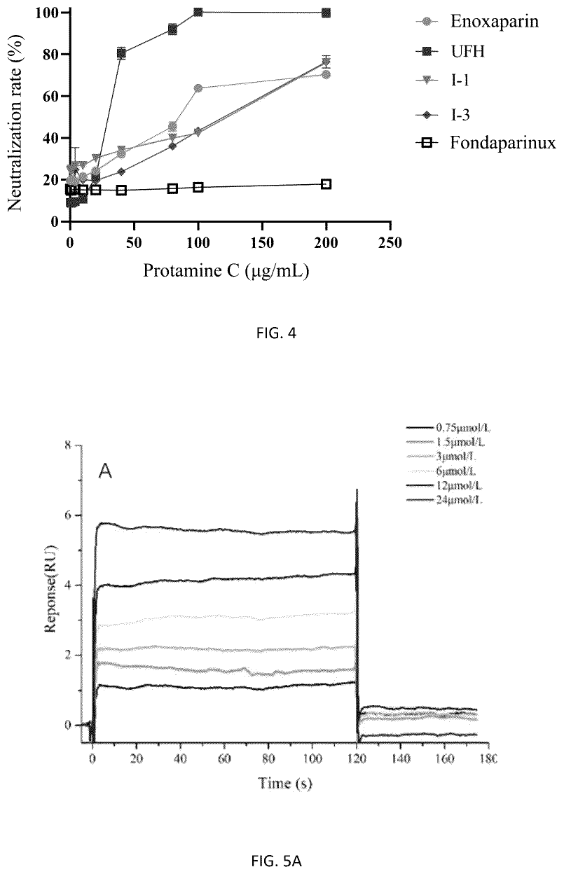

The effects of different concentrations of protamine on anti-Xa activities of the heparin-chondroitin chimeras I-1 and I-3 prepared in Example 2 and Example 3 were measured by using the chromogenic substrate assay. The results showed that, similar to UFH, the in-vitro anti-FXa activities of the heparin-chondroitin chimeras I-1 and I-3 could be effectively reversed by protamine. The in-vitro anti-Xa activities of the heparin-chondroitin chimeras I-1 and I-3 could be reversed by 75.9% and 76.4% by protamine, as shown in FIG. 4. Therefore, the heparin-chondroitin chimeras I-1 and I-3 prepared in the present disclosure were novel heparin molecules whose anticoagulant activities can be efficiently neutralized by protamine.

### EXAMPLE 6: Evaluation of Risk of HIT Induced by Heparin-Chondroitin Chimeras I-1 and I-3

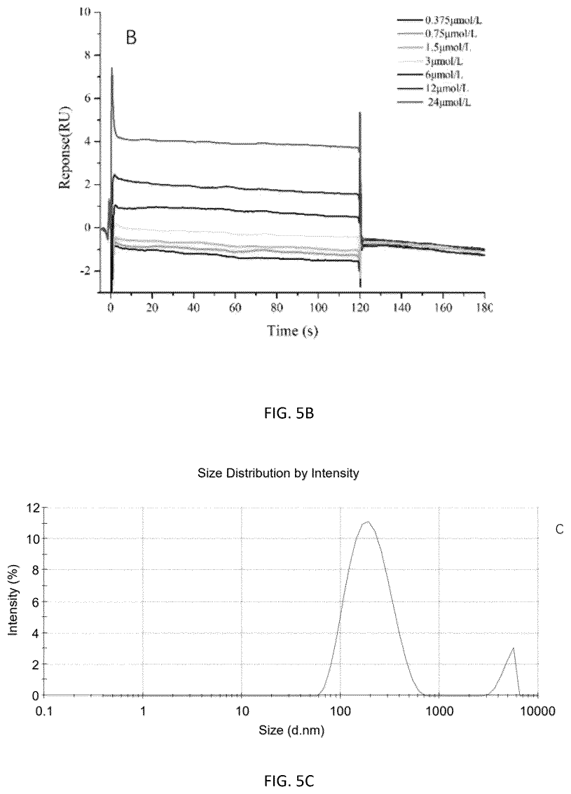

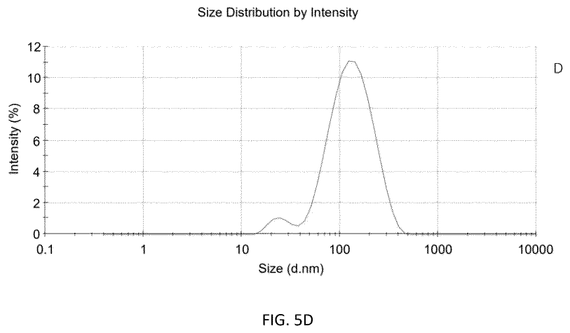

The affinity of each compound to PF4 was determined by using surface plasmon resonance (SPR). Test results were shown in FIG. 5A and FIG. 5B. The equilibrium dissociation constants K_{D}(M) of chimeric molecules I-1 and I-3 from PF4 were calculated as 1.325×10⁻⁵ and 3.181×10⁻⁵, respectively, as well as 5.441×10⁻⁷ for UFH and 5.361×10⁻⁶ for enoxaparin measured under the same conditions. It can thus be seen that the affinity between each compound and the PF4 protein was as follows: UFH>enoxaparin>I-1>I-3. The affinity between I-3 and PF4 was the smallest, indicating that I-3 and PF4 were the least likely to form a stable super-large complex.

Secondary structure changes of PF4 induced by various compounds were evaluated by using circular dichroism (CD), and the content of an antiparallel R lamella was used as an evaluation index of conformational change. The contents of antiparallel R lamellas obtained by mixing chimeric molecules I-1 and I-3 with PF4 at isomolar ratios were 25.4% and 23.5%, respectively, which were lower than that (43%) of enoxaparin, wherein the stability of the complex formed by I-3 and PF4 was the weakest.

The average particle sizes of compounds formed by UFH, enoxaparin, I-1, I-3 and fondaparinux with PF4 were 441.9 nm, 363.2 nm, 202.4, 105.7 nm and 61.57 nm, respectively measured by using a particle size analyzer. The results were shown in FIG. 5C and FIG. 5D. Therefore, the average particle size of the complexes formed by various chimeric molecules and PF4 was smaller than that of enoxaparin, wherein the particle size of the complex formed from I-3 was the smallest.

In summary, the risk of adverse reactions such as fetal HIT caused by the heparin-chondroitin chimera I-1 and I-3 was lower than that of animal-derived UFH and LMWH enoxaparin, which belonged to safer new anticoagulant molecules.

What is claimed is:

1. An anticoagulant heparin-chondroitin chimeric saccharide molecule, formed by connecting heparin-oligosaccharide chains containing an AT-binding sequences in series with a chondroitin-oligosaccharide chain, and being a compound having a structure shown in formula I or a pharmaceutically acceptable salt thereof; 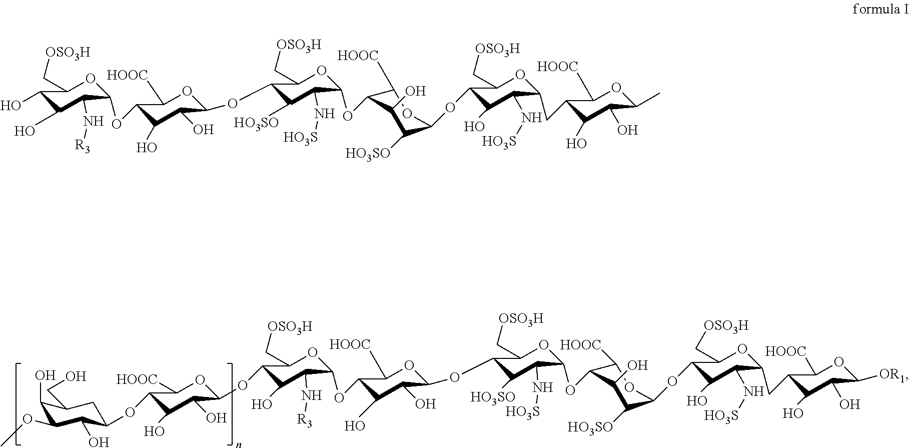
wherein R₁ is phenyl or substituted phenyl, aromatic heterocycle or substituted aromatic heterocycle, or hydrogen (—H), or alkyl and alkylamine-derived groups with characteristic ultraviolet absorption;
R₂ and R₃ are sulfonic acid groups (—SO₃H) or acetyl groups (—COCH₃); and
n is 1-4.

2. The anticoagulant heparin-chondroitin chimeric saccharide molecule according to claim 1, wherein the anticoagulant heparin-chondroitin chimeric saccharide molecule is selected from any of the following compounds or pharmacologically acceptable salts thereof: 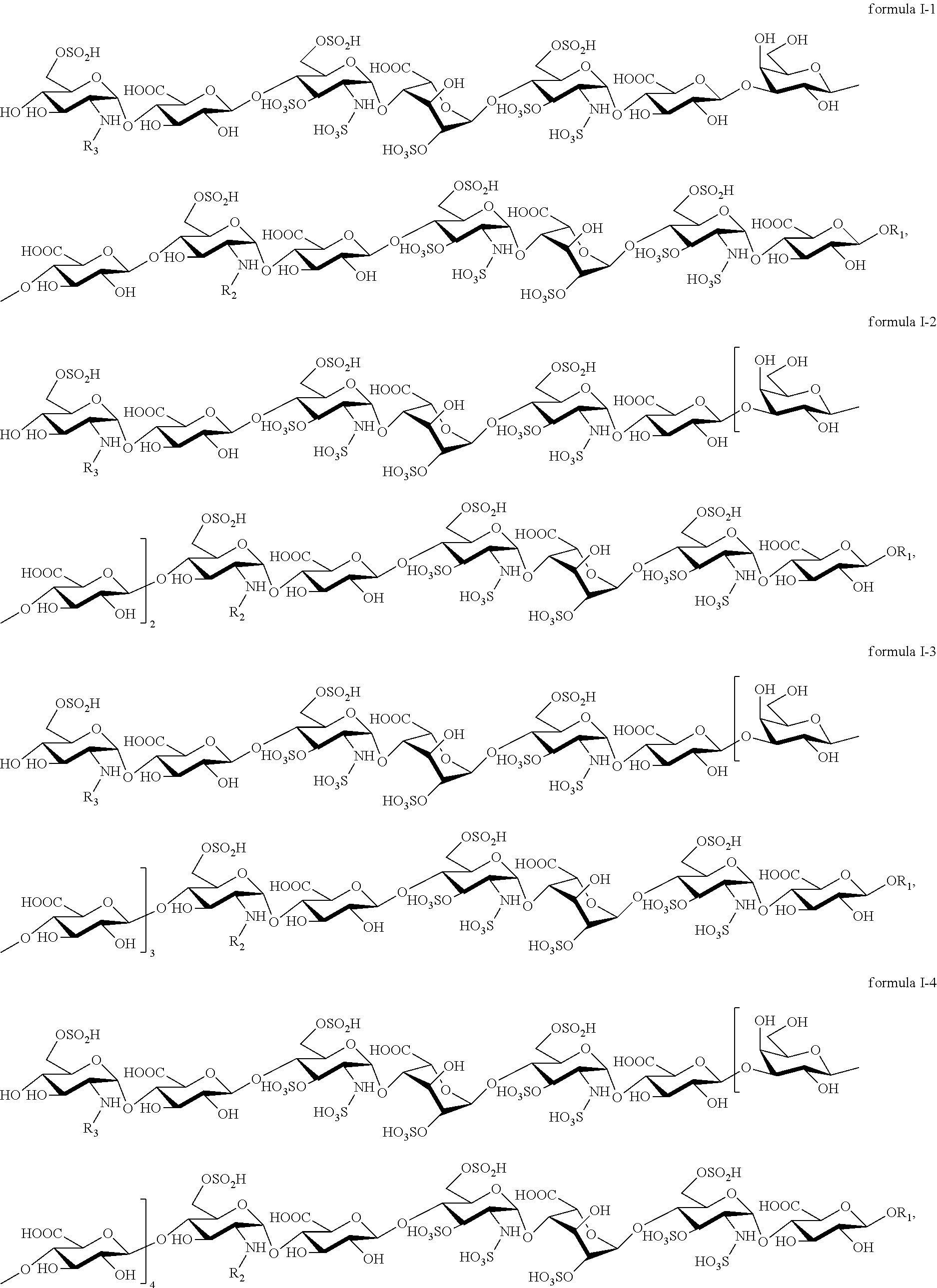
in formulas I-1 to I-4, R₂ and R₃ are sulfonic acid groups (—SO₃H) or acetyl groups (—COCH₃).

3. A preparation method of the anticoagulant heparin-chondroitin chimeric saccharide molecule according to claim 1, carried out by a chemical enzymatic synthesis strategy, wherein the method comprises: by taking a glucuronic acid (GlcA) derivative in which a reducing end is covalently connected to an R₁ group as a starting substrate, the following steps a and b of the saccharide chain extension reactions catalyzed by glycosyltransferases repeating at least once; and in conjunction with two, three or four of steps d, e, f and g of the heparin-saccharide chain chemoenzymatic modification reactions;
at step a, under the catalysis of N-acetylglucosaminyltransferase (KfiA) or Heparosan synthase 2 (PmHS2), with UDP-GlcNTFA or UDP-GlcNAc as a glycosyl donor, a GlcNTFA residue or GlcNAc residue of the glycosyl donor is transferred to a GlcA residue at a non-reducing end of a substrate through an α-1,4 glycosidic bond to obtain an intermediate compound;
at step b, under the catalysis of a PmHS2 enzyme, with UDP-GlcA as a glycosyl donor, a GlcA residue of the glycosyl donor is connected to glucosamine (GlcNTFA or GlcNAc) at the non-reducing end of the substrate through a β-1,4 glycosidic bond to obtain an intermediate compound;
at step c, chondroitin synthase (KfoC) catalysis is performed;
at step d, the heparin intermediate is still placed on ice in a mild alkaline aqueous solution, all GlcNTFA residues of a saccharide chain are subjected to trifluoroacetyl (TFA) removal and converted into GlcNH₂, and then into GlcNS under the catalysis of N-sulfotransferase (NST) to obtain an N-sulfated intermediate;
at step e, under the co-catalysis of C5-isomerase (C₅-epi) and 2-O-sulfotransferase (2OST), a specific GlcA residue in a saccharide chain of the N-sulfated product between two GlcNSs or between GlcNS (non-reducing end) and GlcNAc is converted into 2-O-sulfated iduronic acid (IdoA2S), thereby obtaining an intermediate containing an IdoA2S residue;
at step f, under the co-catalysis of 6-O-sulfotransferase 1 and 6-O-sulfotransferase 3 (6OST1 and 6OST3), 6-OH of all GlcNS or GlcNAc residues of the substrate saccharide chain is subjected to sulfated modification to form GlcNS6S or GlcNAc6S, thereby forming a 6-O-sulfated intermediate; and
at step g, under the catalysis of 3-O-sulfotransferase 1 (3OST1), 3-OH of GlcNS6S in the substrate saccharide chain between GlcA and IdoA2S is sulfated (GlcNS6S3S) to obtain a final target compound.

4. The preparation method according to claim 3, wherein step c comprises the following steps c1 and c2, wherein
at step c1, under the catalysis of chondroitin synthase KfoC, with UDP-GalNAc as a glycosyl donor, a GalNAc residue of the glycosyl donor is transferred to a GlcA residue at the non-reducing end of the saccharide chain substrate through a 3-1,4 glycosidic bond to obtain an intermediate compound; and
at step c2, under the catalysis of chondroitin synthase KfoC, with UDP-GlcA as a glycosyl donor, a GlcA residue of the glycosyl donor is transferred to a GalNAc residue at the non-reducing end of the saccharide chain substrate through a 3-1,3 glycosidic bond to obtain an intermediate compound.

5. The preparation method according to claim 3, wherein the starting substrate is p-nitrophenyl-β-D-glucuronide (GlcA-PNP); at step a, N-acetylglucosaminyltransferase (KfiA) and Heparosan synthase 2 (PmHS2) are recombinantly expressed in Escherichia coli; KfiA is derived from Escherichia coli K5; PmHS2 is derived from Pasteurella multocida; and at step c, chondroitin synthase (KfoC) is recombinantly expressed in Escherichia coli; and KfoC is derived from Escherichia coli K4.

6. The preparation method according to claim 3, wherein at steps a, b and c, a buffer used in an enzyme-catalyzed reaction is 50 mmol/L Tris-HCl; Tris-HCl contains 6 mmol/L MnCl₂ with pH of 7.0-7.5; the reaction temperature is 20° C.-37° C.; the addition amount of enzymes and the substrate and the reaction time are not limited; the obtained enzymatic reaction solution is purified by reversed-phase C18 or anion-exchange column chromatography to obtain an intermediate compound; and the addition amount of the glycosyl donor is more than 1.2 times the equivalent of the substrate;
at steps d, e, f and g, heparin-modifying enzymes NST, C5-epi, 2OST, 6OST1, 6OST3 and 3OST1 are recombinantly expressed by Escherichia coli, yeast or insect cells; heparin-modifying enzymes NST, 2OST, 6OST1, 6OST3 and 3OST1 all use 3′-phosphoadenosine-5′-phosphosulfate (PAPS) as a sulfate-based donor; a buffer catalyzed by each modifying enzyme is 50 mmol/L 2-(N-morpholino)ethanesulfonic acid (MES) with pH of 7.0-7.5; the reaction temperature is 20° C.-37° C.; the addition amount and the reaction time of the enzymes and the heparin intermediate substrate are not limited; the obtained reaction solution is purified by anion-exchange column chromatography; and
at steps d, e, f and g, the addition amount of the sulfate-based donor is 1.2-10 times the equivalent of the substrate.

7. The preparation method according to claim 4, wherein the preparation method is selected from one of the following synthesis routes:
No.: I-1, R₂=—SO₃H, the synthesis route is:
(a→b)×2→d→e→a→b→d→c1→c2→f→(a→b)×2→d→e→a→d, or no→f→g (22/21 steps);
No.: I-1, R₂=—COCH₃, the synthesis route is:
(a→b)×3→c1→c2→(a→b)×2→d→e→a→d, or no→f→g (18/17 steps);
No.: I-2, R₂=—SO₃H, the synthesis route is:
(a→b)×2→d→e→a→b→d→(c1→c2)×2→f→(a→b)×2→d→e→a→d, or no→f→g (24/23 steps);
No.: I-2, R₂=—COCH₃, the synthesis route is:
(a→b)×3→(c1→c2)×2→(a→b)×2→d→e→a→d, or no→f→g (20/19 steps);
No.: I-3, R₂=—SO₃H, the synthesis route is:
(a→b)×2→d→e→a→b→d→(c1→c2)×3→f→(a→b)×2→d→e→a→d, or no→f→g (26/25 steps);
No.: I-3, R₂=—COCH₃, the synthesis route is:
(a→b)×3→(c1→c2)×3→(a→b)×2→d→e→a→d, or no→f→g (22/21 steps);
No.: I-4, R₂=—SO₃H, the synthesis route is:
(a→b)×2→d→e→a→b→d→(c1→c2)×4→f→(a→b)×2→d→e→a→d, or no→f→g (28/27 steps); and
No.: I-4, R₂=—COCH₃, the synthesis route is:
(a→b)×3→(c1→c2)×4→(a→b)×2→d→e→a→d, or no→f→g (24/23 steps).

8. The preparation method according to claim 3, wherein a preparation method of a chimeric octadecasaccharide molecule composed of two heparin hexasaccharide sequences and one chondroitin hexasaccharide sequence comprises the following synthesis route:
(a→b)×2→d→e→a→b→d→(c1→c2)×3→f→(a→b)×2→d→e→a→d→f→g (26 steps); and
the preparation method of a chimeric octadecasaccharide molecule composed of two heparin hexasaccharide sequences and one chondroitin hexasaccharide sequence comprises the following steps:
i) under the catalysis of N-acetylglucosaminyltransferase (KfiA) or Heparosan synthase 2 (PmHS2), with UDP-GlcNTFA as a glycosyl donor, transferring a GlcNTFA residue of the glycosyl donor to GlcA at a non-reducing end of a substrate through an α-1,4 glycosidic bond to obtain a heparin disaccharide backbone intermediate;
ii) under the catalysis of a PmHS2 enzyme, with UDP-GlcA as a glycosyl donor, connecting a GlcA residue of the glycosyl donor to GlcNTFA at a non-reducing end of a disaccharide backbone through a J-1,4 glycosidic bond to obtain a heparin trisaccharide backbone intermediate;
iii) repeating step 1 and step 2) to extend a saccharide chain to obtain a heparin pentasaccharide backbone intermediate;
iv) placing the pentasaccharide backbone intermediate still on ice in a mild alkaline aqueous solution, performing trifluoroacetyl (TFA) removal on all GlcNTFA residues of the saccharide chain, and converting them into GlcNH₂, and then into GlcNS under the catalysis of N-sulfotransferase (NST), thereby obtaining an N-sulfated heparin pentasaccharide intermediate;
v) under the co-catalysis of C5-isomerase (C5-epi) and 2-O-sulfotransferase (2OST), converting a specific GlcA residue in a saccharide chain of the N-sulfated heparin pentasaccharide intermediate between two GlcNSs into 2-O-sulfated iduronic acid (IdoA2S) to obtain a heparin pentasaccharide intermediate containing one IdoA2S residue;
vi) with the heparin pentasaccharide intermediate containing one IdoA2S residue as a substrate, extending the saccharide chain under the catalysis of KfiA or PmHS2 to obtain a heparin hexasaccharide intermediate, with reaction conditions referring to step 1); with the hexasaccharide intermediate as a substrate, extending the saccharide chain into heptasaccharide under the catalysis of PmHS2, thereby obtaining a heparin heptasaccharide intermediate, with reaction conditions referring to step 2);
vii) placing the heparin heptasaccharide intermediate still on ice in a mild alkaline aqueous solution, performing trifluoroacetyl (TFA) removal on all GlcNTFA residues of the saccharide chain, and converting them into G1cNH₂, and then into GlcNS under the catalysis of N-sulfotransferase (NST), thereby obtaining an N-sulfated heparin heptasaccharide intermediate, with reaction conditions referring to step 4);
viii) under the catalysis of chondroitin synthase (KfoC), with UDP-GalNAc as a glycosyl donor, transferring a GalNAc residue of the glycosyl donor to GlcA at the non-reducing end of the substrate through a 3-1,4 glycosidic bond to obtain a heparin-chondroitin chimeric octosaccharide intermediate; under the continuous catalysis of KfoC, with UDP-GlcA as a glycosyl donor, transferring a GlcA residue of the glycosyl donor to a GalNAc residue at a non-reducing end of the chimeric octosaccharide intermediate substrate through a 3-1,3 glycosidic bond, thereby obtaining a heparin-chondroitin chimeric nonasaccharide intermediate;
ix) repeating step 8) twice to introduce a chondroitin hexasaccharide sequence to obtain a heparin-chondroitin chimeric tridecasaccharide intermediate;
x) under the co-catalysis of 6-O-sulfotransferases 1 and 3 (6OST1 and 6OST3), performing sulfated modification on 6-OH of all GlcNS residues of the N-sulfated heparin heptasaccharide intermediate containing one IdoA2S residue to form GlcNS6S, thereby obtaining a 6-O-sulfated heparin-chondroitin chimeric tridecasaccharide intermediate;
xi) referring to step 1) and step 2), extending a saccharide chain of the heparin-chondroitin chimeric tridecasaccharide intermediate to obtain a heparin-chondroitin chimeric pentadecasaccharide intermediate; continuously referring to step 1) and step 2), extending the saccharide chain to obtain a heparin-chondroitin chimeric heptadecasaccharide intermediate containing a heparin backbone saccharide chain at a non-reducing end;
xii) referring to step 4, performing trifluoroacetyl (TFA) removal on all GlcNTFA residues of a heparin chain at a non-reducing end of the heparin-chondroitin chimeric heptadecasaccharide intermediate, and converting them into GlcNH₂, and then into GlcNS under the catalysis of NST to obtain a heparin-chondroitin heptadecasaccharide intermediate containing an N-sulfated heparin chain at a non-reducing end;
xiii) under the co-catalysis of C5-isomerase (C5-epi) and 2-O-sulfotransferase (2OST), converting a specific GlcA residue in a heparin saccharide chain at a non-reducing end in chimeric pentasaccharide between two GlcNSs into IdoA2S, thereby obtaining a heparin-chondroitin chimeric pentasaccharide intermediate containing two IdoA2S residues;
xiv) with UDP-GlcNTFA as a glycosyl donor, extending a saccharide chain under the catalysis of KfiA or PmHS2 to obtain a heparin-chondroitin chimeric octadecasaccharide intermediate, with reaction conditions referring to step 1); referring to step 4, modifying a newly introduced GlcNTFA residue of the chimeric octadecasaccharide intermediate into GlcNS;
xv) under the co-catalysis of 6-O-sulfotransferases 1 and 3 (6OST1 and 6OST3), performing sulfated modification on 6-OH of a GlcNS residue of a heparin saccharide chain at a non-reducing end of the chimeric octadecasaccharide intermediate into GlcNS6S, thereby obtaining a heparin-chondroitin chimeric octadecasaccharide intermediate whose heparin chain is 6-O-sulfated completely; and
xvi) under the catalysis of 3-O-sulfotransferase 1 (3OST1), sulfating 3-OH of a GlcNS6S residue of a heparin saccharide chain in the chimeric octadecasaccharide intermediate between GlcA and IdoA2S to obtain a target compound I-3 formed by connecting two heparin hexasaccharide sequences containing AT-binding sequences with one non-sulfated chondroitin hexasaccharide sequence, wherein R₂ and R₃ are sulfonic acid groups (—SO₃H).

9. An anticoagulant and anti-thrombotic drug, comprising the anticoagulant heparin-chondroitin chimeric saccharide molecule according to claim 1 and one or more pharmaceutically acceptable carriers or excipients.
